(19) United States
(12) Patent Application Publication
(10) Pub. No.: US 2022/0202882 A1
(43) Pub. Date: Jun. 30, 2022

(54) EFFECT OF COMBINATION OF BIFIDOBACTERIUM AND BERBERINE ON PRE-DIABETES AND TYPE 2 DIABETES

(71) Applicants: The Fourth Military Medical University, Xi'an, Shaanxi (CN); Shenzhen Aimigene Technology Co., Ltd., Shenzhen, Guangdong (CN)

(72) Inventors: Qiuhe JI, Xi'an (CN); Xiaokai WANG, Shenzhen (CN); Jie MING, Xi'an (CN); Xiaoqiang XU, Shenzhen (CN); Yan XIA, Shenzhen (CN)

(21) Appl. No.: 17/260,730

(22) PCT Filed: Jul. 16, 2019
(86) PCT No.: PCT/CN2019/096102

§ 371 Date: Jan. 15, 2021

(30) Foreign Application Priority Data
Jul. 17, 2018 (CN) 201810781386.2

(51) Int. Cl. A61K 35/745; A61K 31/4375; A61K 9/19; A61P 3/10; A61K 9/48

(52) U.S. Cl. CPC A61K 35/745; A61K 31/4375; A61K 9/48; A61P 3/10; A61K 9/19

(57) ABSTRACT

Disclosed is the use of Bifidobacterium and berberine in the preparation of a medicament for treating pre-diabetes or type 2 diabetes mellitus. Specifically, the medicament is applicable to pre-diabetic patients, or patients of type 2 diabetes mellitus with fasting plasma glucose (FPG) level being 5.6 mmol/L or more and lower than 8.0 mmol/L, or venous plasma glucose at 2 hours post OGTT being 7.8 mmol/L or more and less than 17 mmol/L.

### FIELD OF THE INVENTION

The present application relates to the combined use of Bifidobacterium and berberine in treatment of pre-diabetes and type 2 diabetes mellitus.

### BACKGROUND OF THE INVENTION

in recent years, the incidence of diabetes in China is increasing year by year with increased obese and overweight population, due to the acceleration of urbanization and aging, along with lifestyle changes. According to the data collected by International Diabetes Federation (IDF), the patients with diabetes in China reached 109 million in 2015, accounting for about 71% of the patients around Western Pacific, which increased by 11.2 million compared to that in 2013, showing rapid growth. The IDF predicts that by 2040, the diabetic patients in China will reach 151 million, with an increase of nearly 50% compared to that of 2015. Further to tumors and cardio-cerebrovascular diseases, diabetes has become the third chronic disease in the world that seriously threatens human health.

Among diabetes, type 2 diabetes mellitus (T2DM) is the main type, accounting for 90% or more of the diabetic patients. The pathogenesis of T2DM is still unclear and what is only known currently is that insulin resistance is the main factor leading to type 2 diabetes mellitus. In clinical treatment, plasma glucose is usually controlled by oral administration of hypoglycemic drugs. However, the use of these drugs may cause varying degrees of adverse drug reactions and side effects. For instance, the use of metformin may lead to a relatively high incidence of gastrointestinal side effects; thiazolidinediones may increase the risk of having cardiovascular diseases; sulfonylureas may cause hypoglycemia; and glucagon-like peptide−1 (GLP-1) analogues may cause adverse gastrointestinal reactions and antibody formations. Insulin, although rather remarkably lowering plasma glucose, needs to be injected into the body and may cause hypoglycemia (Samer El-Kaissi, et al. Pharmacological management of type 2 diabetes mellitus: an update, Current Diabetes Reviews, 2011; 7, 392-405).

More and more researchers believe that gut microbiota is closely related to metabolic diseases such as diabetes. The gut microbe digest food components indigestible for human body and thereby regulate the energy homeostasis of the host (Turnbaugh P J, el al. An obesity-associated gut microbiome with increased capacity for energy harvest. Nature, 2006; 444: 1027-1131; Bäckhed F. et al. The gut microbiota as an environmental factor that regulates fat storage. Proc Natl Acad Sci USA, 2004; 101: 15718-15723). Gut microbiota dysbiosis elevates the circulating endotoxin levels, causing inflammations, and inducing insulin resistance, obesity, and even diabetes (Cani P D, et al. Changes in gut microbiota control metabolic endotoxemia-induced inflammation in high-fat diet-induced obesity and diabetes in mice. Diabetes, 2008; 57:1470-1481; Cani P D, et al. Metabolic endotoxemia initiates obesity and insulin resistance. Diabetes, 2007; 56: 1761-1772). Through dietary intervention, the structure of the gut microbiota can be adjusted and symptoms of diabetes can be greatly relieved. For instance, prebiotics are able to selectively increase the number of Bifidobacterium in the intestines of mice with high-fat diet, reduce intestinal toxins, and significantly improve glucose tolerance and anti-inflammatory factor secretions (Cani A M et al. Selective increases of Bifidobacterium in gut microflora improve high-fat-diet-induced diabetes in mice through a mechanism associated with endotoxaemia, Diabetologia, 2007; 50:2374-2383).

Berberine is a kind of alkaloid, which can be extracted from plants such as Coptis chinensis, Phellodendri cortex chinensis, Berberis julianae, etc. It has significant antibacterial effect and is often used clinically to treat intestinal infections, especially bacterial diarrhea. In recent years, studies have further showed that berberine can play a role in the treatment of diabetes by affecting the gut microbiota. For instance, berberine is shown to eliminate insulin resistance in high-fat diet-fed rats, and to a certain extent reverse the changes of gut microbiota caused by high-fat diet (Xu Zhang, et al. Structural changes of gut microbiota during berberine-mediated prevention of obesity and insulin resistance in high-fat diet-fed rats, Plos One, 2012; 7(8):e42529). After administering newly diagnosed patients of type 2 diabetes mellitus with berberine, some species of Bifidobacteria genus in the gut increased or decreased, and body mass index (BMI), fasting plasma glucose, and glycosylated hemoglobin etc., dropped significantly (Lingling Chen, el al. Berberine ameliorates type 2 diabetes via modulation of Bifidobacterium species, tumor necrosis factor alpha, and lipopolysaccharide, Int J Clin Exp Med, 2016; 9(6):9365-9372).

Probiotics are active microorganisms that provide health benefits to the host when administered in sufficient amounts. Numerous studies and clinical trials have shown that the regulation of gut microbiota by probiotics, especially by Lactobacillus and Bifidobacterium, may effectively prevent the occurrence of and manage diabetes (Homayouni-Rad A, et al. Diabetes management by probiotic: current knowledge and future perspective. Int J Vitam Nutr Res. 2017, Apr. 24: 1-13). Studies also suggested that Bifidobacterium supplementation reduced endotoxins and inflammatory cytokines, and thus improved high-fat-diet induced glucose tolerance and glucose-induced insulin secretion in mice (Cani P D. et al. Selective increases of Bifidobacterium in gut microflora improve high-fat-diet induced diabetes in mice through a mechanism associated with endotoxaemia Diabetologia 2007; 50: 2374-83).

No studies or clinical trials have yet been directed to the combined use of probiotics and berberine in treatment of type 2 diabetes mellitus or pre-diabetes.

### SUMMARY OF THE INVENTION

The present application relates to a composition for treatment of pre-diabetes or type 2 diabetes mellitus, comprising probiotics and berberine. The composition of the present application may be, for instance, a compound capsule comprising probiotics and berberine, or a combination of individually packaged probiotics and berberine. The probiotics may be lyophilized powder of the probiotics, and berberine may be a berberine-containing tablet.

The probiotics may be Bifidobacterium, such as B. adolescentis, e.g., B. adolescentis DM8504.

The composition may comprise 50,000,000 to 1,000,000,000 viable probiotics such as Bifidobacterium. In some embodiments, the composition may comprise 50,000,000 or more, 100,000,000 or more, 150,000,000 or more, 200,000,000 or more, 250,000,000 or more, 300,000,000 or more, 350,000,000 or more, or 400,000,000 or more, and 1,000,000,000 or less, 900,000,000 or less, 800,000,000 or less, 700,000,000 or less, 600,000,000 or less, or 500,000,000 or less viable probiotics such as Bifidobacterium. In some embodiments, the composition may comprise 50,000,000, 100,000,000, 150,000,000, 200,000,000, 250,000,000, 300,000,000, 350,000,000, 400,000,000, 500,000,000, 600,000,000, 700,000,000, 800,000,000, 900,000,000 or 1,000,000,000 viable probiotics such as Bifidobacterium.

The composition may comprise 0.5-2 g berberine. In some embodiments, the composition may comprise berberine of 0.5 g or more, 0.75 g or more, or 1 g or more, and 2 g or less, 1.75 g or less, or 1.5 g or less. In one embodiment, the composition may comprise berberine of 0.5 g, 0.75 g, 1 g, 1.5 g, 1.75 g, or 2 g.

In one embodiment, the composition comprises a berberine-containing tablet having 0.5 g berberine, and lyophilized Bifidobacterium powder having 100,000,000 viable Bifidobacterium.

On another aspect, the present application provides the use of probiotics such as Bifidobacterium and berberine in preparation of a composition for treatment of pre-diabetes or type 2 diabetes mellitus. In one embodiment, the composition of the present disclosure is a compound capsule containing probiotics such as Bifidobacterium, and berberine. The probiotics may be lyophilized probiotics powder, and berberine may be a berberine-containing tablet. In one embodiment, the compound capsule contains a berberine-containing tablet having 0.5 g berberine, and lyophilized Bifidobacterium powder having 100,000,000 viable Bifidobacterium.

The present application further provides a method for treating pre-diabetes or type 2 diabetes mellitus, comprising administering a subject in need thereof an therapeutically effective amount of probiotics such as Bifidobacterium and berberine.

The daily dose of probiotics such as Bifidobacterium may be 50,000,000 to 1,000,000,000 viable probiotics. In some embodiments, the daily dose of probiotics such as Bifidobacterium may be 50,000,000, or more, 100,000,000 or more, 150,000,000 or more, 200,000,000 or more, 250,000,000 or more, 300,000,000 or more, 350,000,000 or more, or 400,000,000 or more, and 1,000,000,000 or less, 900,000,000 or less, 800,000,000 or less, 700,000,000 or less, 600,000,000 or less, or 500,000,000 or less viable probiotics. In some embodiments, the daily dose of probiotics such as Bifidobacterium may be 50,000,000, 100,000,000, 150,000,000, 200,000,000, 250,000,000, 300,000,000, 350,000,000, 400,000,000, 500,000,000, 600,000,000, 700,000,000, 800,000,000, 900,000,000 or 1,000,000,000 viable probiotics.

The daily dose of berberine may be 0.5-2 g. In some embodiments, the daily dose of berberine may be 0.5 g or more, 0.75 g or more, or 1 g or more, and 2 g or less, 1.75 g or less, or 1.5 g or less. In one embodiment, the daily dose of berberine may be 0.5 g, 0.75 g, 1 g, 1.5 g, 1.75 g, or 2 g.

Bifidobacterium and berberine may be concurrently administered, or administered separately over a given interval. The combined administration may be administered twice a day, more than twice a day, or less than twice a day, as needed.

The composition of the present application is applicable to patients with pre-diabetes or type 2 diabetes mellitus. In some embodiments, the patients with type 2 diabetes mellitus are those with fasting plasma glucose (FPG) level being 5.6 mmol/L or more and lower than 8.0 mmol/L, or venous plasma glucose at 2 hours post OGTT being 7.8 mmol/L or more and less than 17 mmol/L.

When Bifidobacterium is administered with berberine, the fasting plasma glucose (FPG), 2-hour postprandial blood glucose (2 h-PPG), glycated hemoglobin (HbA1c), total cholesterol and low-density lipoprotein (LDL) can be lowered in people newly diagnosed with abnormal blood glucose (including those with pre-diabetes and type 2 diabetes mellitus), with effectiveness efficacy comparable to or slightly better than that of berberine alone or Bifidobacterium alone. The combined use is also with fewer side effects compared to berberine alone, e.g., with milder diabetic autonomic neuropathy symptoms characterized by alternating constipation and diarrhea. In addition, the combined use can significantly reduce urine ketone body level, which has not been observed in the berberine or Bifidobacterium monotherapy group, indicating that the combined use may alleviate the diabetic acidosis in subjects to some extent.

### DETAILED DESCRIPTION OF THE INVENTION

Oral hypoglycemic drugs may effectively lower blood glucose level and have been widely used as first-line treatment in China. However, the use of these drugs brings as well varying degrees of adverse drug reactions and side effects. For example, the use of metformin follows a higher incidence of gastrointestinal side effects; thiazolidinediones increase the risk of having cardiovascular disease; sulfonylureas cause hypoglycemia; and glucagon-like peptide-1 (GLP-1) analogues cause gastrointestinal reactions and antibody formations. On the other hand, insulin injection, which has rather remarkable effects in lowering blood glucose, may cause hypoglycemia. Further, injection is less convenient than oral administration.

Berberine is a kind of alkaloid, commonly in the form of berberine hydrochloride, with molecular formula of C₂₀H₁₈ClNO₄. Berberine has a certain modification effects on diabetes, causing fewer adverse drug reactions and side effects compared to the commonly used oral hypoglycemic drugs. Berberine exerts mild modification effects on abnormal blood lipid levels (Hui Dong, et al. Berberine in the treatment of type 2 diabetes mellitus: a systemic review and meta-analysis, 2012; 591654), and is one of the best candidates for the treatment of type 2 diabetes mellitus. The main adverse drug reaction and side effect caused by berberine is mild intestinal discomfort, such as constipation.

Bifidobacterium plays a role in balancing gut microbiota, and is added to food in daily life in order to relieve gastrointestinal symptoms such as constipation. Bifidobacterium is highly accepted by its non-drug nature.

In the present application, Bifidobacterium and berberine are used as a combination, for treating pre-diabetes and type 2 diabetes mellitus. The results showed that the effects of the combination, in terms of lowering the fasting plasma glucose, 2-hour postprandial blood glucose, and glycated hemoglobin (HbA1c), are significantly better than that of the placebo or Bifidobacterium alone, and these indicator levels decrease more in the combination group than in the berberine group. In addition, the effects of the combination in terms of lowering the total cholesterol and low-density lipoprotein are significantly better than that of the placebo, and comparable to berberine alone and Bifidobacterium alone. Besides, the combined use of Bifidobacterium and berberine can significantly lower the ketone body level in urine, which was not observed in neither the berberine group nor the Bifidobacterium group.

Compared with the placebo, the combined use of Bifidobacterium and berberine does not evidently increase side effects, indicating higher safety level than other diabetes drugs. Compared with berberine alone, the combined use in the present application causes fewer side effects, e.g., with fewer incidence of constipation, gastrointestinal reaction, and diabetic autonomic neuropathy symptoms characterized by alternating constipation and diarrhea.

Bifidobacterium and berberine were once clinically used in combination for the treatment of diarrhea, and were separately dosed with a given interval because of the antibacterial effect of berberine (Li, Youchao, Clinical efficacy of berberine combined with triple viable Bifidobacterium in the treatment of chronic diarrhea, Flight Surgeon, 2016, Issue 23). In the present application, the inventors have observed comparable effectiveness no matter Bifidobacterium and berberine were administered at the same time or separately over a given interval. In the trial as described in the Examples, Bifidobacterium and berberine were separately administered with a given interval for better convenience and compliance. Nevertheless, Bifidobacterium and berberine may be prepared in compound capsules. That is, one capsule can be divided into two separate cells, and Bifidobacterium and berberine can be separately loaded in the two cells. The subjects or patients, when swallowing the capsules, can take both ingredients. In addition to the compound capsules, Bifidobacterium and berberine may be prepared in other compound forms, as long as the two ingredients are separately contained in one carrier/container.

In the present application, probiotics such as Bifidobacterium may be in form of lyophilized powder for easier storage at room temperature. Probiotics may also be in other forms such as being distributed in culture media provided with appropriate storage and transportation conditions. In addition, although only the effect of Bifidobacterium on treatment of pre-diabetes and type 2 diabetes mellitus has been demonstrated in the present application, other probiotics that can regulate gut microbiota to lower the blood glucose of pre-diabetes or diabetic patients can also be combined with berberine to treat pre-diabetes, type 2 diabetes mellitus or other types of diabetes, such as Lactobacillus acidophilus, Lactobacillus brevis, Lactobacillus casei. Lactobacillus crispatus, Lactobacillus germanica, Lactobacillus fermentum, Lactobacillus helveticus, Lactobacillus johnsonii, Lactobacillus paracasei, Lactobacillus plantarum, Lactobacillus reuteri. Lactobacillus rhamnosus, Lactobacillus salivarius, Lactococcus lactis, Bifidobacterium animalis, Bifidobacterium bifidum, Bifidobacterium breve, Bifdobaclerium longum, and Bifidobacterium hornis.

Berberine may be in the form of tablets, or other forms such as granules, capsules etc. Different berberine forms may require different daily dose, which may be determined by the physicians or pharmacists according to the actual condition. When the composition of the present application is administered to different populations, the physicians or pharmacists may determine applicable doses according to the gender, age, medical history, disease development, etc.

“Fasting plasma glucose” herein refers to the blood glucose level of blood samples collected after overnight fasting (at least 8-10 hours without any intakes but water), which is the most commonly used indicator for diabetes. Fasting plasma glucose level over 5.6 mmol/L is considered hyperglycemic, according to the American Diabetes Association criteria.

“Glycated hemoglobin” herein refers to the hemoglobin in the red blood cells bonded with the saccharides in the serum, formed through a slow, sustaining and irreversible glycation reaction. The glycated hemoglobin level depends on the concentration of blood glucose and the contact duration of glucose and hemoglobin, and is not related to the time when the blood is collected, whether the patient is on fast, or whether insulin is dosed, etc. Glycated hemoglobin can effectively reflect the blood glucose in diabetics over the past 1-2 months. Glycated hemoglobin consists of HbA1a, HbA1b and HbA1c, while HbA1c with stable structure accounts for about 70% and thus is used as a monitoring indicator for diabetes control.

“Total cholesterol” refers to the overall cholesterols contained in all lipoproteins in the blood, which is the sum of lipoprotein-bound cholesterols and free cholesterols. The total cholesterol level mainly depends on genetic factors and lifestyles. The cholesterol level perfect for adults as recommended by national and foreign experts is lower than 5.2 mmol/L.

“Low-density lipoprotein” refers to the lipoprotein granule that transfers cholesterols to cells in the peripheral tissue, which may be oxidized to the oxidized low-density lipoprotein. When low-density lipoproteins, especially oxidized low-density lipoproteins (OX-LDL), are excessive, the cholesterols they are carrying may accumulate on artery walls and may likely to cause arteriosclerosis over time. Low-density lipoproteins are the primary atherogenic lipoproteins among all serum lipoproteins and therefore known as the “bad cholesterols”.

‘Urine ketone’ refers to the ketone body present in the urine, consisting of 3 intermediate lipid metabolism products in the body, i.e., acetone, acetoacetic acid, and β-hydroxybutyric acid. During starvation, when glucose metabolism is impaired, lipolysis increases, and diabetic acidosis occurs, the ketone bodies are produced at a higher rate than they are utilized by human body, resulting in ketonuria. The administration of biguanides, a kind of hypoglycemic drug that inhibits cellular respiration, may cause ketonuria with lowered blood glucose. “Diabetic ketoacidosis” or “diabetic acidosis” refers to ketosis caused by reduced glucose utilization and increased ketone body production by breaking down fats.

### Example 1. Treatment of Pre-Diabetes and Type 2 Diabetes Mellitus Using Bifidobacterium and/or Berberine

From June 2015 to October 2018, a double-blind, randomized, and parallel-controlled trial, studying the effects of the combined use of Bifidobacterium and berberine on pre-diabetic and type 2 diabetes mellitus patients, was conducted in 10 research centers in Xi'an, Yan'an and Shangluo of Shaanxi province, etc., including the First Affiliated Hospital of the Fourth Military Medical University.

The inclusion criteria of the trial are as follows:

1) Aged 18-70 years;

2) Body Mass Index (BMI) between 19-30 kg/m²;

3) Diagnosed with impaired glucose tolerance or type 2 diabetes mellitus (5.6≤fasting plasma glucose (FPG)<8.0 mmol/L at V1 stage; 6.1≤fasting plasma glucose (FPG)<8.0<mmol/L or 7.8≤2-hour postprandial venous blood glucose (2 h-PPG)<17 mmol/L at V2 stage, please see V1 and V2 in Table 1 below);

4) Having not participated in any other clinical trials over the past three months; and

5) Having given their informed consent.

The exclusion criteria for the trial are as follows:

1) Type 1 diabetes mellitus;

2) Diabetics, previously treated or not, with FPG equal to or higher than 8 mmol/L or 2-h PPG equal to or higher than 17 mmol/L;

3) Pregnant or nursing female subjects, or female subjects intending to get pregnant or taking no effective contraception methods;

4) Patients with impaired liver functions, specifically with aspartate transaminase (AST) or alanine transaminase (ALT) readings more than twice the normal upper limit;

5) Patients with impaired kidney functions, with blood creatinine equal to or higher than 133 μmol/L;

6) Subjects who had been treated but with uncontrolled severe hypertension, or subjects with severe hypertension who had not been treated (systolic pressure equal to or higher than 160 mmHg and/or diastolic pressure equal to or higher than 95 mmHg);

7) Subjects with history of chronic gastrointestinal disease or gastrointestinal surgery.

8) Subjects with history of cancer or cancer treatment history;

9) Subjects with any clinically diagnosed diseases that were believed to interfere with the results of this trial;

10) Subjects with incomplete intelligence, mental illness, unwillingness to participate in the study or language problems (including inability to read and write);

11) Subjects who were known or suspected to be allergic to the drugs or related products in this study; and

12) Subjects who were known or suspected to be with alcohol abuse, drug abuse or illicit drug abuse.

The trial included an enrollment stage of 2 weeks and a treatment stage of 16 weeks. After passing the first visit 1 (see V1 in Table 1), the subjects entered the enrollment stage, i.e., 2 weeks of lifestyle intervention, followed by a re-screening (see V2 in Table 1). The subjects were subjected to a 16-week course of treatment after passing the screening. See Table 1 for the detailed screening parameters.

After the enrollment stage, the baseline data of the 300 successfully enrolled subjects were collected. Meanwhile, the subjects started to receive lifestyle guidance, and were randomly divided into four groups for 16 weeks of treatments.

1) Bifidobacterium group: the subjects were administered with Bifidobacterium capsules (Livzon Pharmaceutical Group Inc., batch no.: 20141013), 0.35 g/capsule, each capsule containing 50,000,000 viable bacteria (Bifidobacterium adolescentis DM8504), 2 capsules/time, 2 times/day, plus berberine placebo tablets (Northeast Pharma Group Shenyang First Pharma Co., Ltd., Lot no.: 20150101, containing 80.2% microcrystalline cellulose, 9.4% pregelatinized starch, 6.4% sodium carboxymethyl starch, 1% magnesium stearate, and 3% copovidone), 0.1 g/tablet, 5 tablets/time, 2 times/day.

2) Berberine group: the subjects were administered with berberine tablets (Northeast Pharma Group Shenyang First Pharma Co., Ltd., Lot no.: 130917, each tablet weighted 0.165 g and contained 0.1 g active berberine ingredient (berberine hydrochloride), with the same excipients as the placebo tablets), 5 tablets/time, 2 times/day, plus Bifidobacterium placebo capsules (Livzon Pharmaceutical Group Inc., Lot no.: 141001, specifically containing 0.348 g spray-dried lactose, and 0.002 g magnesium stearate), 0.35 g/capsule, 2 capsules/time, 2 times/day.

3) Combination Group: the subjects were administered with Bifidobacterium capsules, 0.35 g/capsule, each capsule containing 50,000,000 viable bacteria (B. adolescentis DM8504), 2 capsules/time, 2 times/day, plus berberine tablets, 0.1 g/tablet, each tablet containing 0.1 g active berberine ingredient (berberine hydrochloride), 5 tablets/time, 2 times/day.

4) Control group: the subjects were administered with Bifidobacteria placebo capsules, 0.35 g/capsule, 2 capsules/time, 2 times/day, plus berberine placebo tablets, 0.1 g/tablet, 5 tablets/time, 2 times/day.

Bifidobacterium capsules were taken after meals and berberine tablets were taken before meals.

During the study, subjects were allowed to take drugs that were necessary for safety and health and not explicitly excluded by the study, which must be documented though. The use of any hypoglycemic drugs, and the oral administration or intravenous injection of corticosteroids for more than 7 days were prohibited or restricted during the study.

In addition, from the beginning of the enrollment stage, subjects followed the diet and exercise guidance for proper diet and exercise, with total calorie intake of 30 kcal/kg ideal body weight/day, including 20% carbohydrates (providing 55% of calories), 40% fats (providing 25% of calories), and 40% proteins (providing 20% of calories).

Before blood collection, the subjects were kept still for 5 min. For serum insulin assays, 5 ml of whole blood was collected at fasting, and 30 min and 2 h post glucose intake, and then subjected to centrifugation for 2 h. The obtained serum was collected in the cryogenic vials. For the glycosylated hemoglobin (HbA1c) test, 2 ml of whole blood was collected and placed in EDTA-containing tubes. For the GLP-1 and glucagon measurements, 5 ml of whole blood was collected in vacutainers containing EDTA and aprotinin at fasting, and 30 min and 2 h post meals, subjected to centrifugation, and the obtained serum was collected in cryogenic vials and stored at −20° C. Soybean-sized stool sample was placed in a dry and sterile collection tube, and sent to the detection center within 24 h for cryopreservation.

Before the oral glucose tolerance test (OGTT), the subjects took regular diets for at least 3 days, with carbohydrates no less than 150 g. After 10-12 h fasting, initial blood samples were collected after the subjects emptied their bladders. Then the subjects drank a sugar solution made by dissolving 75 g anhydrous glucoses in 300 ml water over about 5 min, and were collected for blood samples after 30 min and 2 h.

Tests of all samples in the study were carried out in a designated laboratory with standardized criteria. Blood analysis covered hundreds of indicators including insulin, amino acids, cytokines etc.

TABLE 1
Arrangements and screening parameters during enrollment stage and treatment stage

| | Study Phases | | | | | | |
|---|---|---|---|---|---|---|---|
| | Screening | | | | | | |
| | stage | Treatment stage | | | | | |
| | Visit no. (to the office) | | | | | | |
| | | V2 | Telephone | | | | |
| | V1 | Randomization | interview | V3 | V4 | V5 | V6 |
| Visit time window | −2 w ± 7 d | 0 w (baseline) | 2 w ± 7 d | 4 w ± 7 d | 8 w ± 7 d | 12 w ± 7 d | 16 w ± 7 d |
| Population data/baseline | | | | | | | |
| Informed consent | ✓ | | | | | | |
| Inclusion/exclusion criteria | ✓ | ✓ | | | | | |
| Population data | ✓ | | | | | | |
| Physical examination | ✓ | ✓ | | ✓ | ✓ | ✓ | ✓ |
| Vital signs | ✓ | ✓ | | ✓ | ✓ | ✓ | ✓ |
| Complications & combined | ✓ | ✓ | ✓ | ✓ | ✓ | ✓ | ✓ |
| medication | | | | | | | |
| Lifestyle guidance | ✓ | ✓ | ✓ | ✓ | ✓ | ✓ | ✓ |
| Treatment stage | | | | | | | |
| Diet Survey | | ✓ | | ✓ | ✓ | ✓ | ✓ |
| Distribution of blood-glucose | | ✓ | | | | | |
| meter | | | | | | | |
| Distribution and collection of | | ✓ | | ✓ | ✓ | ✓ | ✓ |
| research diary | | | | | | | |
| Distribution of medications | | ✓ | | ✓ | ✓ | ✓ | |
| Collection and count of | | | | ✓ | ✓ | ✓ | ✓ |
| medications | | | | | | | |
| Related medications | | ✓ | | ✓ | ✓ | ✓ | ✓ |
| Safety evaluation | | | | | | | |
| Pregnancy test (urine test) | ✓ | ✓ | | ✓ | ✓ | ✓ | ✓ |
| Physical examination | ✓ | ✓ | | ✓ | ✓ | ✓ | ✓ |
| Vital signs | ✓ | ✓ | | ✓ | ✓ | ✓ | ✓ |
| 12-lead electrocardiogram | ✓ | ✓ | | ✓ | | | ✓ |
| Blood and urine routine tests | ✓ | ✓ | | ✓ | | | ✓ |
| Liver function test | ✓ | ✓ | | ✓ | | | ✓ |
| Kidney function test | ✓ | ✓ | | ✓ | | | ✓ |
| Blood lipid profile | ✓ | ✓ | | ✓ | | | ✓ |
| Adverse drug reaction | ✓ | ✓ | ✓ | ✓ | ✓ | ✓ | ✓ |
| Efficacy assessment | | | | | | | |
| Fasting plasma glucose | ✓ | ✓ | | ✓ | ✓ | ✓ | ✓ |
| Glycosylated hemoglobin | | ✓ | | | | | ✓ |
| (HbA1c) | | | | | | | |
| OGTT test (BG. insulin) | | ✓ | | | | | ✓ |
| SMBG | | | | ✓ | ✓ | ✓ | ✓ |
| Body weight and BMI | ✓ | ✓ | | ✓ | ✓ | ✓ | ✓ |
| Other assessments | | | | | | | |
| Gut microbiota | | ✓ | | | | | ✓ |
| Gut hormone (0, 30 min, 2 h) | | ✓ | | | | | ✓ |
| Backup samples | | | | | | | |
| Stool sample | | ✓ | | | | | ✓ |
| Blood sample | | ✓ | | | | | ✓ |
| Anticoagulated blood sample | | ✓ | | | | | ✓ |

OGTT: oral glucose tolerance test;
BG: blood glucose;
SMBG: self-monitoring of blood glucose;
BMI: body mass index
At V2, the inclusion criteria for fasting plasma glucose and OGTT were: 5.6 ≤ fasting plasma glucose (FPG) < 8.0 mmol/L or 7.8 ≤ venous plasma glucose at 2 hours post OGTT (2-h PPG) < 17 mmol/L.

The four groups were numbered A. B, C, and D throughout the trial and analysis, and unblinded after all analysis was completed.

After the sample testing was finished, 1) Chi-Squared test and t-test were used to evaluate the intergroup balance of the baseline data including the demographic characteristics and pre-treatment indicators, wherein the baseline data referred to those collected at the second visit, before the formal administration, the data collected at the first visit on the enrollment stage were used instead if the data at the second visit was lacking; 2) two-way disordered CM-CH11, Kruska-Wilcox test, etc. were used for analysis and intergroup comparison of pre- and post-treatment indicators such as fasting plasma glucose (FPG), HbA1c, plasma glucose at 2 hours post OGTT (2-h PPG), blood pressure, blood lipids, body weight and BMI; 3) adverse events and hypoglycemia events were described in terms of the type, frequency and relevance with the drugs, for safety evaluation. Software SAS 9.1.3 was used for statistical analysis with all analysis processes being programmed.

Among the 300 subjects enrolled, 50 were in the combination group, 100 in the placebo group, 50 in the berberine group and 100 in the Bifidobacterium group, wherein 256 subjects completed the trial, 40, 90, 39 and 87 respectively for the groups.

All subjects who followed the intent-to-treat principle, grouped randomly, and received the treatment trial and at least one efficacy evaluation were included in the full Analysis Set (FAS). This set was analyzed for demographic characteristics, pre-treatment baselines, medical history, efficacy and safety.

Subjects who completed the treatment trial according to the protocol without significant deviation from it, and finished all evaluations were included in the Per Protocol Set (PPS). The inclusion criteria of PPS at least included meeting the inclusion criteria specified in the trial protocol, experiencing all planned visits and completing the CRF, no medications or treatments during the trial that may affect the efficacy and evaluation, and good compliance (80-120%). The PPS was analyzed for the demographic characteristics, pre-treatment baselines, medical history and efficacy.

Demographic characteristics mainly included gender and ethnicity. Pre-treatment baseline indicators included body weight, BMI, systolic/diastolic pressure, pulse, breath, waist circumference, hip circumference, and electrocardiogram. Medical history included allergy, smoking and alcohol drinking history. Efficacy analysis was mainly focused on body weight, BMI, fasting plasma glucose, glycosylated hemoglobin (HbA1c), blood glucose response, metabolic changes, etc.

The analysis indicated the intergroup balance in terms of demographic characteristics and past medical history without significant differences, but group differences regarding certain pre-treatment baseline indicators such as the pre-treatment total cholesterol levels were found.

### Example 2. Combined Use of Bifidobacterium and Berberine Lowered Blood Glucose and Lipids

Multiple indicators before and after the subjects took Bifidobacterium plus berberine, berberine alone, Bifidobacterium alone, and placebo were summarized in Table 2 below, and certain indicators' pairwise comparison results were summarized in Table 3.

From the data, it can be seen that the pre-treatment fasting plasma glucose (FPG) showed no group difference, but the post-treatment fasting plasma glucose tended to be different among groups (with P value of 0.0733 for the FAS). Accordingly, the differences between post-treatment FPG and pre-treatment FPG were different among groups. Kruskal-Wilcox test showed that both the combined use of berberine and Bifidobacterium, and berberine alone, lowered the fasting plasma glucose level significantly. If the FPG levels in the two groups were compared to that in the placebo group, FPG decreased more in the combination group. Bifidobacterium alone had no evident effect.

Pre-treatment glycosylated hemoglobin (HbA1c) levels showed no intergroup difference, while post-treatment HbA1c tended to be different among groups, and the HbA1c changes during treatment were different among groups. According to the Kruskal-Wilcox test, the combined use of berberine and Bifidobacterium significantly lowered the glycosylated hemoglobin level, compared with the placebo and Bifidobacterium alone. The effect of berberine only was not significantly different from that of the combined use of berberine and Bifidobacterium (with P value of 0.2399). However, when comparing these 2 groups with the placebo group, the combination group showed slightly better efficacy. Bifidobacterium alone had no glycosylated hemoglobin lowing effect.

In terms of total cholesterol, there were group differences both before and after treatment, so the level change was compared among groups. Kruskal-Wilcox test showed that both the combined use of berberine and Bifidobacterium, and berberine alone can significantly reduce the total cholesterol level, with berberine only having slightly better effect. Bifidobacterium alone did not have evident total cholesterol lowing effect.

There was no difference among groups regarding pre-treatment low-density lipoprotein, while the post-treatment low-density lipoprotein and low-density lipoprotein change during the trial showed intergroup differences. According to the Kruskal-Wilcox test, the post-treatment low-density lipoprotein level in the combination group was significantly lower than that in the placebo group and the Bifidobacterium alone group, and lower than that in the berberine alone group as well (P=0.0710). In addition, in terms of the low-density lipoprotein level change during the trial, the low-density lipoprotein lowering effect of the combination group or the berberine group was better than that of the placebo group (with P value of 0.1027 for both).

In terms of urine ketone body level, the combined use of Bifidobacterium and berberine significantly reduced ketone levels in urine, indicating the effect of the combination on alleviating diabetic acidosis in diabetic patients, which was not observed in the berberine only group or the Bifidobacterium only group.

The venous plasma glucose at 2 hours post meals (2-h PPG) showed no difference among groups prior to the treatment, but with intergroup differences after treatment. Accordingly, 2 h-PPG change during the trial showed differences among groups. According to Kruskal-Wilcox test, the combined use of Bifidobacterium and berberine can significantly reduce 2 h-PPG, berberine only had good effect too with P value of 0.1008, but Bifidobacterium alone had no such effect. There was no significant difference between the combination group and the berberine only group. However, according to the comparison between these groups and the placebo group, the combination group might have slightly better effects. Besides, the proportion of subjects with post-treatment 2 h-PPG lower than 10.0 mmol/L showed differences among groups.

The proportion of subjects with post-treatment HbA1c lower than 7.0% differed or tended to differ among groups (P value of 0.0685 for FAS, and P value of 0.0360 for PPS), while the proportion of subjects with post-treatment HbA1c lower than 7.0% and no hypoglycemia showed no significant differences among groups.

Other indicators showed no group differences, such as post-treatment HbA1c <6.0% (two-way disordered CMH-CHI #), post-treatment HbA1c <6.0% plus no hypoglycemia (two-way disordered CMH-CHI #), post-treatment FPG <6.1 mmol/L (two-way disordered CMH-CHI #), post-treatment FPG <7.2 mmol/L (two-way disordered CMH-CHI #), post-treatment 2 h-PPG <7.8 mmol/L (two-way disordered CMH-CHI #), post-treatment HbA1c decrease by 0.5% (two-way disordered CMH-CHI #), systolic pressure change (Wilcoxon rank sum test), diastolic pressure change (Wilcoxon rank sum test), triglyceride change (Analysis of Variance), BMI change (Analysis of Variance) and body weight change (Analysis of Variance).

According to above analysis results, the combined use of Bifidobacterium and berberine can significantly reduce the fasting plasma glucose level, plasma glucose level at 2 hours post meals, glycosylated hemoglobin level, total cholesterol level and low-density lipoprotein level, with effects comparable to that of berberine or Bifidobacterium alone, or slightly better than that of berberine only. In addition, the combined use of berberine and Bifidobacterium can significantly reduce the subjects' ketone levels in urine, indicating its effect on alleviating diabetic acidosis, which was not observed when Bifidobacterium or berberine was used alone.

TABLE 2
Physiological indicators before and after taking Bifidobacterium plus berberine, berberine, Bifidobacterium or placebos

| | FAS | | | | | | | | | | |
|---|---|---|---|---|---|---|---|---|---|---|---|
| Physiological | | Combination | Placebo | Berberine | Bifidobacterium | Analysis | | | | | |
| indicators | | group | group | group | group | approach/P value | PPS | | | | |
| Pre-treatment | N (drop out) | 50(0) | 100(0) | 50(0) | 100(0) | Kruskal-Wallis | | | | | |
| FPG (mmol/L) | Mean value ± | 6.4200 ± | 6.4100 ± | 6.4700 ± | 6.4000 ± | test, P = 0.9040 | | | | | |
| | SD | 0.7620 | 0.7390 | 0.6580 | 0.6630 | | | | | | |
| Post-treatment | N (drop out) | 41(9) | 92(8) | 41(9) | 88(12) | Kruskal-Wallis | | | | | |
| FPG (mmol/L) | Mean value ± | 6.1300 ± | 6.6300 ± | 6.1300 ± | 6.4300 ± | test, P = 0.0733 | | | | | |
| | SD | 0.9150 | 1.3300 | 0.7880 | 0.9700 | | | | | | |
| Post-treatment | N (drop out) | 41(9) | 92(8) | 41(9) | 88(12) | Kruskal-Wallis | | | | | |
| FPG - pre- | Mean value ± | −0.3760 ± | 0.2220 ± | −0.2450 ± | 0.0282 ± | test, P = 0.0191 | | | | | |
| treatment FPG | SD | 1.0500 | 1.1700 | 0.7590 | 0.8980 | | | | | | |
| (mmol/L) | | | | | | | | | | | |
| Post-treatment | N (drop out) | 49(0) | 99(0) | 49(0) | 100(0) | Two-way | 39(0) | 86(0) | 37(0) | 83(0) | Two-way |
| HbA1c <7.0% | Yes-N (%) | 47(95.92) | 87(87.88) | 48(97.96) | 88(88.00) | disordered | 38(97.44) | 74(86.05) | 36(97.30) | 71(85.54) | disordered |
| | | | | | | CMH-CHI # | | | | | CMH-CHI # |
| | | | | | | P = 0.0685 | | | | | P = 0.0360 |
| Post-treatment | N (drop out) | 49(0) | 99(0) | 49(0) | 100(0) | Two-way | 39(0) | 86(0) | 37(0) | 83(0) | Two-way |
| HbA1c <7.0% | Yes-N (%) | 40(81.63) | 75(75.76) | 40(81.63) | 76(76.00) | disordered | 31(79.49) | 62(72.09) | 28(75.68) | 61(73.49) | disordered |
| with no | | | | | | CMH-CHI # | | | | | CMH-CHI # |
| hypoglycemia | | | | | | P = 0.7349 | | | | | P = 0.8080 |
| Pre-treatment | N (drop out) | 50(0) | 100(0) | 50(0) | 100(0) | Kruskal-Wallis | | | | | |
| 2-h PPG | Mean value ± | 11.2 ± | 11.6 ± | 11.2 ± | 11.4 ± | test, P = 0.9320 | | | | | |
| (mmol/L) | SD | 2.35 | 3.07 | 2.38 | 3.08 | | | | | | |
| Post-treatment | N (drop out) | 41(9) | 91(9) | 40(10) | 88(12) | Kruskal-Wallis | | | | | |
| 2-h PPG | Mean value ± | 9.79 ± | 11.5 ± | 9.84 ± | 11.3 ± | test, P = 0.0044 | | | | | |
| (mmol/L) | SD | 2.49 | 3.66 | 2.93 | 3.5 | | | | | | |
| Post- treatment | N (drop out) | 41(9) | 91(9) | 40(10) | 88(12) | Kruskal-Wallis | | | | | |
| 2-h PPG - pre- | Mean value ± | −1.5 ± | −0.0801 ± | −1.1 ± | −0.246 ± | test, P = 0.0200 | | | | | |
| treatment 2-h | SD | 2.59 | 3.05 | 2.66 | 3.41 | | | | | | |
| PPG (mmol/L) | | | | | | | | | | | |
| Pre-treatment | N (drop out) | 50(0) | 100(0) | 50(0) | 99(1) | Kruskal-Wallis | | | | | |
| HbA1c (%) | Mean value ± | 6.1300 ± | 6.220 ± | 6.1900 ± | 6.1900 ± | test, P = 0.9410 | | | | | |
| | SD | 0.7170 | 0.6770 | 0.5270 | 0.5890 | | | | | | |
| Post-treatment | N (drop out) | 41(9) | 91(9) | 41(9) | 89(11) | Kruskal-Wallis | | | | | |
| HbA1c (%) | Mean value ± | 5.9000 ± | 6.1400 ± | 6.0800 ± | 6.1500 ± | test, P = 0.1350 | | | | | |
| | SD | 0.5170 | 0.6600 | 0.5200 | 0.6140 | | | | | | |
| Post-treatment | N (drop out) | 41(9) | 91(9) | 41(9) | 88(12) | Kruskal-Wallis | | | | | |
| HbA1c - pre- | Mean value ± | −0.2560 ± | −0.0835 ± | −0.1170 ± | −0.0545 ± | test, P = 0.0102 | | | | | |
| treatment | SD | 0.3990 | 0.5630 | 0.4680 | 0.5040 | | | | | | |
| HbA1c (%) | | | | | | | | | | | |
| Post-treatment | N (drop out) | 49(0) | 99(0) | 49(0) | 100(0) | Two-way | 39(0) | 86(0) | 37(0) | 83(0) | Two-way |
| 2-h PPG <10.0 | Yes-N (%) | 32(65.31) | 46(46.46) | 35(71.43) | 45(45.00) | disordered | 23(58.97) | 37(43.02) | 24(64.86) | 31(37.35) | disordered |
| mmol/L | | | | | | CMH-CHI # | | | | | CMH-CHI # |
| | | | | | | P = 0.0027 | | | | | P = 0.0138 |
| Pre-treatment | N (drop out) | 49(1) | 100(0) | 50(0) | 100(0) | Kruskal-Wallis | | | | | |
| total cholesterol | Mean value ± | 4.4400 ± | 4.7100 ± | 5.0200 ± | 4.7600 ± | test, P = 0.0364 | | | | | |
| (mmol/L) | SD | 0.9510 | 0.8740 | 1.1400 | 1.0100 | | | | | | |
| Post-treatment | N (drop out) | 42(8) | 92(8) | 41(9) | 89(11) | Kruskal-Wallis | | | | | |
| total cholesterol | Mean value ± | 4.1400 ± | 4.7900 ± | 4.6400 ± | 4.8100 ± | test, P = 0.0018 | | | | | |
| (mmol/L) | SD | 0.9380 | 0.9430 | 1.1500 | 1.0300 | | | | | | |
| Post-treatment | N (drop out) | 41(9) | 92(8) | 41(9) | 89(11) | Kruskal-Wallis | | | | | |
| total cholesterol - | Mean value ± | −0.2590 ± | 0.0930 ± | −0.2770 ± | 0.0219 ± | test, P = 0.0054 | | | | | |
| pre-treatment | SD | 1.1300 | 0.7600 | 0.7250 | 0.6670 | | | | | | |
| total cholesterol | | | | | | | | | | | |
| (mmol/L) | | | | | | | | | | | |
| High-density | N (drop out) | 41(8) | 92(7) | 41(8) | 89(11) | Variance analysis, | 38(1) | 86(0) | 37(0) | 83(0) | Variance |
| lipoprotein | Mean value ± | 0.03 ± | 0.11 ± | 0.00 ± | 0.03 ± | P = 0.0220 | 0.04 ± | 0.11 ± | −0.00 ± | 0.03 ± | analysis, |
| change | SD | 0.21 | 0.23 | 0.23 | 0.21 | | 0.22 | 0.23 | 0.23 | 0.21 | P = 0.0295 |
| (mmol/L) | | | | | | | | | | | |
| Pre-treatment | 49(1) | 100(0) | 50(0) | 100(0) | 49(1) | Kruskal-Wallis | | | | | |
| low-density | 2.69 ± | 2.81 ± | 2.99 ± | 2.85 ± | 2.69 ± | test, P = 0.2700 | | | | | |
| lipoprotein | 0.84 | 0.786 | 0.879 | 0.802 | 0.84 | | | | | | |
| (mmol/L) | | | | | | | | | | | |
| Post-treatment | 42(8) | 92(8) | 41(9) | 89(11) | 42(8) | Kruskal-Wallis | | | | | |
| low-density | 2.41 ± | 2.85 ± | 2.76 ± | 2.9 ± | 2.41 ± | test, P = 0.0032 | | | | | |
| lipoprotein | 0.76 | 0.851 | 0.805 | 0.812 | 0.76 | | | | | | |
| (mmol/L) | | | | | | | | | | | |
| Post-treatment | 41(9) | 92(8) | 41(9) | 89(11) | 41(9) | Kruskal-Wallis | | | | | |
| low-density | −0.241 ± | 0.0396 ± | −0.15 ± | −0.00247 ± | −0.241 ± | test, P = 0.0657 | | | | | |
| lipoprotein - | 0.844 | 0.606 | 0.564 | 0.6 | 0.844 | | | | | | |
| pre-treatment | | | | | | | | | | | |
| low-density | | | | | | | | | | | |
| lipoprotein | | | | | | | | | | | |
| (mmol/L) | | | | | | | | | | | |
| Pre-treatment | N (drop out) | 49(1) | 99(1) | 49(1) | 100(0) | Kruskal-Wallis | | | | | |
| ketone level in | Mean value ± | 0.2240 ± | 0.0404 ± | 0.0000 ± | 0.1100 ± | test, P = 0.0686 | | | | | |
| urine | SD | 0.7150 | 0.2440 | 0.0000 | 0.4470 | | | | | | |
| Post-treatment | N (drop out) | 42(8) | 90(10) | 39(11) | 88(12) | Kruskal-Wallis | | | | | |
| ketone level in | Mean value ± | 0.0000 ± | 0.0111 ± | 0.0513 ± | 0.0341 ± | test, P = 0.3510 | | | | | |
| urine | SD | 0.0000 | 0.1050 | 0.2230 | 0.2370 | | | | | | |
| Post-treatment | N ( drop out) | 41(9) | 89(11) | 38(12) | 88(12) | Kruskal-Wallis | | | | | |
| ketone level in | Mean value ± | −0.2680 ± | −0.0337 ± | 0.0526 ± | −0.0909 ± | test, P = 0.0223 | | | | | |
| urine - pre- | SD | 0.7750 | 0.2800 | 0.2260 | 0.5170 | | | | | | |
| treatment ketone | | | | | | | | | | | |
| level in urine | | | | | | | | | | | |

TABLE 3
Pairwise comparison of physiological indicator changes after taking berberine
plus Bifidobacterium, berberine, Bifidobacterium or placebos

| | | FAS | | | | | |
|---|---|---|---|---|---|---|---|
| | | Combination | Combination | Combination | Placebo | Placebo | Berberine |
| | Statistical | group vs. | group vs. | group vs. | group vs. | group vs. | group vs. |
| Physiological | analysis | Placebo | Berberine | Bifidobacterium | Berberine | Bifidobacterium | Bifidobacterium |
| indicators | method | group | group | group | group | group | group |
| Post-treatment FPG - | Wilcoxon | 0.0045 | 0.4741 | 0.0120 | 0.0357 | 0.5526 | 0.0894 |
| pre-treatment FPG | | | | | | | |
| Post-treatment HbA1c - | Wilcoxon | 0.0134 | 0.2399 | 0.0096 | 0.3710 | 0.6770 | 0.2699 |
| pre-treatment HbA1c | | | | | | | |
| Post-treatment total | Wilcoxon | 0.0401 | 0.5595 | 0.2161 | 0.0095 | 0.2451 | 0.0641 |
| cholesterol - pre-treatment | | | | | | | |
| total cholesterol | | | | | | | |
| Post-treatment low-density | Wilcoxon | 0.0029 | 0.0710 | 0.0029 | 0.5012 | 0.7952 | 0.4682 |
| lipoprotein | | | | | | | |
| Post-treatment low-density | Wilcoxon | 0.1027 | 0.9364 | 0.3118 | 0.1027 | 0.3118 | 0.3118 |
| lipoprotein - pre-treatment | | | | | | | |
| low-density lipoprotein | | | | | | | |
| Post-treatment 2-h PPG - | Wilcoxon | 0.0352 | 0.6820 | 0.0352 | 0.1008 | 0.9837 | 0.1008 |
| pre-treatment 2-h PPG | | | | | | | |
| Post-treatment ketone level | Wilcoxon | 0.0779 | 0.0178 | 0.2079 | 0.2079 | 0.3791 | 0.0779 |
| in urine - pre-treatment | | | | | | | |
| ketone level in urine | | | | | | | |

### Example 3. Combined Use of Bifidobacterium and Berberine Provided Good Safety

Throughout the trial, adverse events, including abnormal bowel movement, occurred in all groups. However, no particularly severe adverse events were observed in any groups.

In the combination group, 118 adverse events were reported in 40 subjects out of 49, with event rate of 81.63%. In the placebo group, 239 adverse events occurred in 82 subjects out of 99, with event rate of 82.83%. In the berberine group, III adverse events were observed in 40 subjects out of 49, with event rate of 81.63%. In the Bifidobacterium group, among the 100 subjects, 203 adverse events were reported in 84 subjects, with event rate of 8400%. According to Fisher's exact test, there was no statistically significant differences in terms of event rate among groups (P=0.9733).

In addition, adverse events correlated with medications in the study such as abdominal discomfort and abnormal bowel movement occurred for 28 times in 15 subjects from the combination group, with the event rate of 30.61%. In the placebo group, 37 correlated adverse events were reported in 26 subjects, with event rate of 26.26%. In the berberine group, 19 events were observed in 9 subjects, with event rate of 18.37%, and 30 events occurred in 21 subjects from the Bifidobacterium group with event rate of 21.0)%. The rate of adverse events correlated with medications in the berberine group was relatively low, but no statistically significant intergroup differences were found using Fisher's exact test (P=0.4298).

It can be seen from the above data and analysis that no severe adverse events occurred in any groups, and that the combined use of Bifidobacterium and berberine did not significantly increase side effects compared with the placebos and is thus safer than other diabetes drugs.

1. A method for treating type 2 diabetes mellitus or pre-diabetes, comprising administering a subject in need thereof a pharmaceutically effective amount of Bifidobacterium and berberine.

2. The method of claim 1, wherein the berberine is a berberine-containing tablet.

3. The method of claim 1, wherein the Bifidobacterium is lyophilized Bifidobacterium powder.

4. The method of claim 1, wherein the Bifidobacterium is Bifidobacterium adolescentis.

5. The method of claim 4, wherein the Bifidobacterium is Bifidobacterium adolescentis DM8504.

6. The method of claim 1, wherein the berberin is administered at a daily dose of 0.5-2 g.

7. The method of claim 6, wherein the berberine is administered at a daily dose of 1 g.

8. The method of claim 1, wherein the Bifidobacterium is administered at a daily dose of 50,000,000 to 1,000,000,000 viable Bifidobacterium.

9. The method of claim 8, wherein the Bifidobacterium is administered at a daily dose of 200,000,000 viable Bifidobacterium.

10. The method of claim 1, wherein the subject is of pre-diabetes.

11. The method of claim 1, wherein the subject is of type 2 diabetes mellitus, with fasting plasma glucose (FPG) level being 5.6 mmol/L or more and lower than 8.0 mmol/L, or with venous plasma glucose at 2 hours post OGTT (2-h PPG) being 7.8 mmol/L or more and less than 17 mmol/L.

12. The method of claim 1, wherein the Bifidobacterium and the berberine are concurrently administered, or administered separately over a given interval.

13. The method of claim 1, wherein the berberine is administered twice a day.

14. The method of claim 1, wherein the Bifidobacterium is administered twice a day.

15. The method of claim 1, wherein the Bifidobacterium and the berberine are contained in a compound capsule.

16. The method of claim 1, wherein the Bifidobacterium and the berberine are orally administered.
